(19) United States
(12) Patent Application Publication
(10) Pub. No.: US 2024/0010370 A1
(43) Pub. Date: Jan. 11, 2024

(54) SPOON AND CHOPSTICK AUTOMATIC PACKAGING APPARATUS

(71) Applicant: BYMTECH CO., LTD., Daejeon (KR)

(72) Inventor: Byoung Yong KIM, Daejeon (KR)

(73) Assignee: BYMTECH CO., LTD., Daejeon (KR)

(21) Appl. No.: 18/346,781

(22) Filed: Jul. 4, 2023

(30) Foreign Application Priority Data
Jul. 8, 2022 (KR) 10-2022-0084650

(51) Int. Cl. B65B 5/06; B65B 55/16; B65B 65/00; A61L 2/10

(52) U.S. Cl. CPC B65B 5/067; B65B 55/16; B65B 65/003; A61L 2/10; A61L 2202/11

(57) ABSTRACT

Proposed is a spoon and chopstick automatic packaging apparatus. The apparatus includes a spoon tray supply portion supplying a spoon tray in which a spoon is mounted in a groove formed in a spoon shape, a chopstick tray supply portion supplying a chopstick tray in which chopsticks are aligned and contained, a spoon moving portion sending down the spoon so that the spoon waits at a spoon entrance, a chopstick moving portion sending down the chopsticks so that the chopsticks wait at a container belt automatic opening and closing portion, a rotary type inspection portion in which the spoon and the chopsticks are placed, an image analysis portion checking a sanitary condition of the spoon and a direction of the chopsticks, a packaging portion packaging the spoon and the chopsticks in a separate packaging bag, and a packaging bag storage portion in which the packaging bag is stored.

### CROSS REFERENCE TO RELATED APPLICATION

The present application claims priority to Korean Patent Application No. 10-2022-0084650, filed Jul. 8, 2022, the entire contents of which are incorporated herein for all purposes by this reference.

### BACKGROUND OF THE INVENTION

### Field of the Invention

The present disclosure relates to a spoon and chopstick automatic packaging apparatus. More particularly, the present disclosure relates to an apparatus capable of automatically packaging a spoon and chopsticks in a packaging bag that includes an advertisement.

### Description of the Related Art

Generally, spoons and chopsticks are utensils used to eat food, and can be reused since the spoons and the chopsticks are made of a metal material.

In restaurants, such spoons and chopsticks are washed after use, and are placed in a spoon and chopstick container so as to be reused. However, since the spoons and the chopsticks contained in the spoon and chopstick container pass through the hands of many people, there is a problem that such spoons and chopsticks are not hygienic and people who use the spoons and the chopsticks are vulnerable to infection with infectious diseases.

In order to solve this problem, in restaurants, spoons and chopsticks are washed and then disinfected with hot water, and are packaged in a packaging bag so as to be reused.

However, there is a problem that manual packaging of each spoon and chopsticks in a packaging bag requires a lot of labor.

### SUMMARY OF THE INVENTION

Accordingly, the present disclosure has been made keeping in mind the above problems occurring in the related art, and an objective of the present disclosure is to provide a spoon and chopstick automatic packaging apparatus device capable of drying a spoon and chopsticks with hot air blowers, then sterilizing the spoon and the chopsticks with UV LEDs, and then automatically and individually packaging the spoon and the chopsticks in a packaging bag that includes an advertisement.

In order to achieve the above objective, according to the present disclosure, there is provided a spoon and chopstick automatic packaging apparatus, the apparatus including: a spoon tray supply portion configured to supply a spoon tray in which a spoon is mounted in a groove formed in a spoon shape; a chopstick tray supply portion configured to supply a chopstick tray in which chopsticks are aligned and contained; a spoon moving portion configured to send down the spoon of the spoon tray so that the spoon waits at a spoon entrance; a chopstick moving portion configured to send down the chopsticks of the chopstick tray so that the chopsticks wait at a container belt automatic opening and closing portion; a rotary type inspection portion in which the spoon and the chopsticks are placed through the spoon moving portion and the chopstick moving portion; an image analysis portion configured to check a sanitary condition of the spoon placed in the rotary type inspection portion and to check a direction of the chopsticks placed in the rotary type inspection portion; a packaging portion configured to package the spoon and the chopsticks in a separate packaging bag; and a packaging bag storage portion in which the packaging bag capable of containing the spoon and the chopsticks is stored.

According to the present disclosure, the spoon and chopstick automatic packaging apparatus may further include first and second hot air blowers, the first and second air blowers being respectively provided on opposite side surfaces of the spoon tray supply portion and the chopstick tray supply portion and being respectively configured to dry the spoon and the chopsticks contained in the spoon tray and the chopstick tray.

According to the present disclosure, the spoon and chopstick automatic packaging apparatus may further include first and second ultrasonic vibrators, the first and second ultrasonic vibrators being respectively provided on lower surfaces of the spoon tray supply portion and the chopstick tray supply portion and being respectively configured to spray sterilizing solutions of first and second sterilizing solution storage containers in a mist type to the spoon and the chopsticks in the spoon tray and the chopstick tray.

According to the present disclosure, the spoon moving portion may include: a first hydraulic automatic opening and closing portion configured to send down the spoon of the spoon tray to a rotation frame portion; the rotation frame portion configured to send down the spoon one by one to a guide portion by a first control portion so as to prevent the spoon from being overturned or reversed; and a spoon entrance which is provided at a lower end of the guide portion and at which the spoon waits.

According to the present disclosure, a first UV LED may be mounted on a passage of the guide portion, and the spoon may be sterilized by the first UV LED.

According to the present disclosure, the spoon and chopstick automatic packaging apparatus may further include a second UV LED, the second UV LED being mounted on an inner side surface of the chopstick tray supply portion storage portion and being configured to sterilize the chopsticks contained in the chopstick tray.

According to the present disclosure, the chopstick moving portion may include: a second hydraulic automatic opening and closing portion configured to send down the chopsticks sterilized of the chopstick tray to a chopstick rotation portion; the chopstick rotation portion configured to align the chopsticks passing through the second hydraulic automatic opening and closing portion and to send down the chopsticks in a line while rotating the chopsticks to prevent mixing of the chopsticks; a chopstick automatic opening and closing portion configured to be closed immediately after dropping one pair of chopsticks; a chopstick storage portion configured to send the dropped chopsticks in line with a compartment of a container rotated; and the container belt automatic opening and closing portion at which the chopsticks moved through the container wait.

According to the present disclosure, when the spoon and the chopsticks that are respectively waiting at the spoon entrance and the container belt automatic opening and closing portion are placed in the rotary type inspection portion, the spoon and the chopsticks may be fixed by a fixation hydraulic portion.

According to the present disclosure, after whether the chopsticks placed in the rotary type inspection portion are positioned forward or reversed is determined, a rotation portion may be rotated to align the chopsticks so that the chopsticks are capable of being moved in a correct direction, then the chopsticks may be sent to the packaging portion when the image analysis portion determines that the chopsticks are in a normal state, and the chopsticks may be sent to a defective product collection box when the image analysis portion determines that the chopsticks are in an abnormal state.

According to the present disclosure, a sanitary condition of the spoon may be checked through the image analysis portion, then the spoon may be sent to the packaging portion when the sanitary condition is good, and the spoon may be sent to a defective product collection box when the sanitary condition is poor.

According to the present disclosure, the image analysis portion may be configured to photograph foreign substances on the spoon by a camera, and may be configured to determine the good and poor conditions by using artificial intelligence.

According to the present disclosure, at least one of a logo, a restaurant advertisement, and a corporate advertisement may be printed on a front surface and a rear surface of the packaging bag.

According to the present disclosure, an individual packaging performed in the packaging portion may be performed such that the packaging bag stored in the packaging bag storage portion is sent down and is stacked on a cover portion, and when one sheet of the packaging bag is sent down through a packaging bag opening and closing portion, the packaging bag may be caught on a packaging bag fixing portion, and when air is blown by an air compressor and an opening of the packaging bag is opened and then a set of the spoon and the chopsticks is dropped into the packaging bag and packaged, the set of the spoon and the chopsticks may be contained in a spoon and chopstick set storage container.

According to the present disclosure, the spoon and chopstick automatic packaging apparatus may further include an operation portion configured such that a spoon and chopstick packaging quantity, a reservation of spoon and chopstick packaging quantity, a UV LED operation time, and a hot air blower operation time may be selected on a touch display and a packaging bag quantity may be displayed on the touch display.

As described above, according to the present disclosure, the spoons and the chopsticks are dried with the hot air blowers and then sterilized with the UV LEDs. Therefore, there is an advantage that infectious diseases may be prevented and clean hygiene may be realized, and also an advertising effect may be realized since the spoons and the chopsticks are automatically and individually packaged in the packaging bag that includes the advertisement.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a plan view illustrating a spoon and chopstick automatic packaging apparatus according to the present disclosure;

FIG. 2 is a side view illustrating the spoon and chopstick automatic packaging apparatus according to the present disclosure;

FIG. 3 is a front view illustrating the spoon and chopstick automatic packaging apparatus according to the present disclosure;

FIG. 4 is a perspective view illustrating the spoon and chopstick automatic packaging apparatus according to the present disclosure; and

FIG. 5 is a view illustrating a front surface and a rear surface of a packaging bag that includes advertisements according to the present disclosure.

### DETAILED DESCRIPTION OF THE INVENTION

Hereinafter, an embodiment of the present disclosure will be described in detail with reference to the accompanying drawings such that a person having ordinary knowledge in the technical field to which the present disclosure belongs may easily implement the embodiment. It is understood that the present disclosure is able to be implemented in various different forms and is not limited to the embodiment described herein.

Then, an exemplary embodiment of a spoon and chopstick automatic packaging apparatus according to the present disclosure will be described.

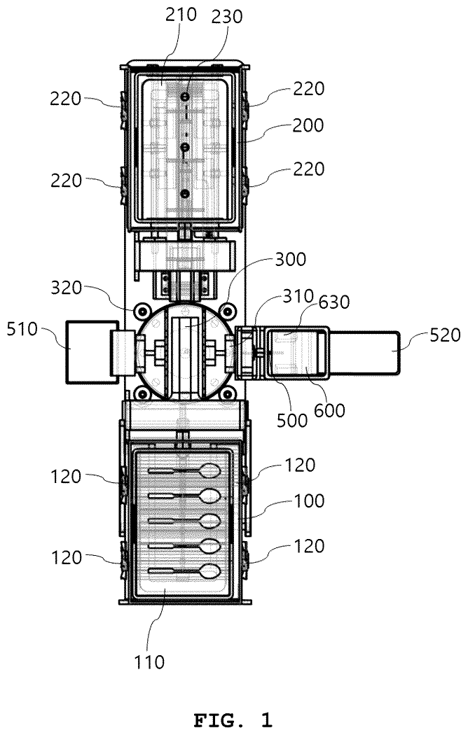

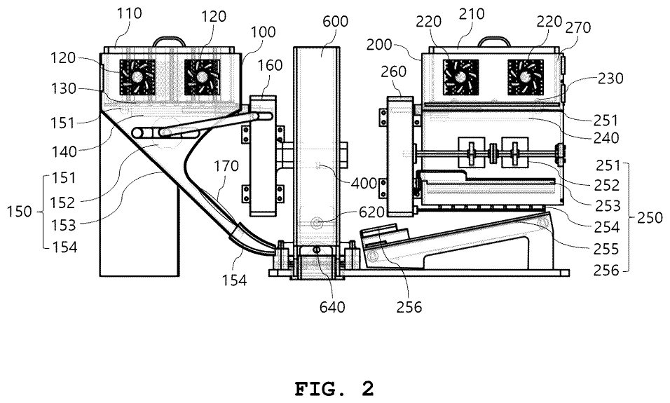

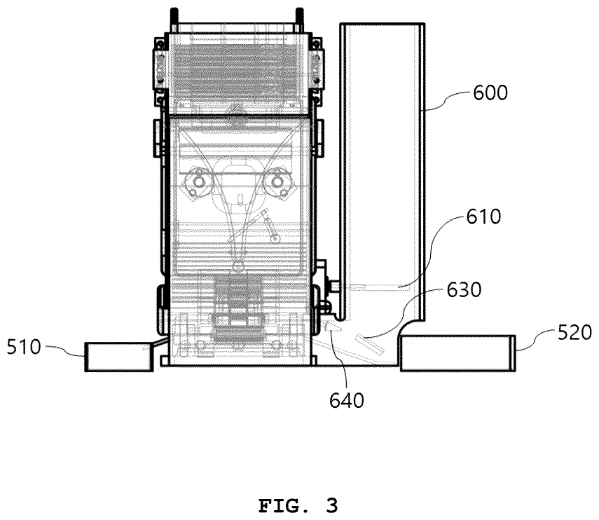

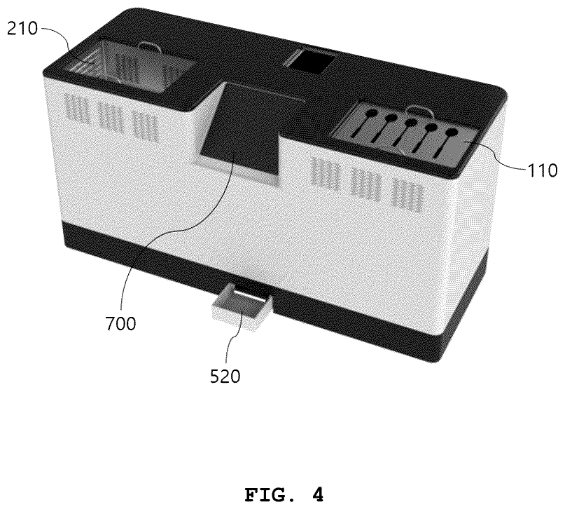

FIG. 1 is a plan view illustrating a spoon and chopstick automatic packaging apparatus according to the present disclosure, FIG. 2 is a side view illustrating the spoon and chopstick automatic packaging apparatus according to the present disclosure, FIG. 3 is a front view illustrating the spoon and chopstick automatic packaging apparatus according to the present disclosure, and FIG. 4 is a perspective view illustrating the spoon and chopstick automatic packaging apparatus according to the present disclosure.

Referring to FIGS. 1 to 4, the spoon and chopstick automatic packaging apparatus according to the present disclosure includes a spoon tray supply portion 100, a chopstick tray supply portion 200, first and second hot air blowers 120 and 220, a spoon moving portion 150, a chopstick moving portion 250, a rotary type inspection portion 300, an image analysis portion 400, a packaging portion 500, a packaging bag supply portion 600, and an operation portion 700.

The spoon tray supply portion 100 may supply a spoon tray 110 in which a spoon washed is mounted in a groove formed in a spoon shape.

Two to four of the first hot air blowers 120 are mounted at opposite side surfaces of the spoon tray supply portion 100, and are configured to dry the spoon contained in the spoon tray 110.

A plurality of first ultrasonic vibrators 130 is mounted at a lower surface of the spoon tray supply portion 100, and a sterilizing solution of a first sterilizing solution storage container 140 is sprayed in a mist type to the spoon contained in the spoon tray 110, and then the spoon is dried by the first hot air blowers 120.

The dried spoon of the spoon tray 110 is sent down through the spoon moving portion 150, and waits at a spoon entrance 154.

Here, the spoon moving portion 150 includes a first hydraulic automatic opening and closing portion 151, a spoon rotation frame portion 152, a guide portion 153, and the spoon entrance 154.

The first hydraulic automatic opening and closing portion 151 sends down the dried spoon of the spoon tray 110 to the spoon rotation frame portion 152.

For example, in order to prevent the spoon from being overturned or reversed while the spoon is sent down, the spoon rotation frame portion 152 sends down the spoon one by one to the guide portion 153 by a first control portion 160. At this time, the spoon is sterilized by a first UV LED 170 mounted at a passage of the guide portion 153 and is moved down, and waits at the spoon entrance 154 at a lower end of the guide portion 153.

The chopstick tray supply portion 200 may supply a chopstick tray 210 in which chopsticks washed are aligned and contained.

Two to four of the second hot air blowers 220 are mounted at opposite side surfaces of the chopstick tray supply portion 200, and are configured to dry the chopsticks contained in the chopstick tray 210.

A plurality of second ultrasonic vibrators 230 is mounted at a lower surface of the chopstick tray supply portion 200, and a sterilizing solution of a second sterilizing solution storage container 240 is sprayed in a mist type to the chopsticks contained in the chopstick tray 210, and then the chopsticks are dried by the second hot air blowers 120. Then, the chopsticks are sterilized by a second UV LED 270 mounted at an inner side surface of the chopstick tray supply portion 200.

The sterilized chopsticks of the chopstick tray 210 are sent down through the chopstick moving portion 250, and wait at a container belt automatic opening and closing portion 256.

Here, the chopstick moving portion 250 may include a second hydraulic automatic opening and closing portion 251, a chopstick rotation portion 252, a chopstick automatic opening and closing portion 253, a chopstick storage portion 254, a container 255, and the container belt automatic opening and closing portion 256.

The second hydraulic automatic opening and closing portion 251 sends down the sterilized chopsticks of the chopstick tray 210 to the chopstick rotation portion 252.

Here, the chopstick rotation portion 252 is configured to align the chopsticks passing through the second hydraulic automatic opening and closing portion 251 and to send down the chopsticks in a line while rotating the chopsticks to prevent mixing of the chopsticks.

The chopstick automatic opening and closing portion 253 is configured to be closed immediately after dropping only one pair of chopsticks. At this time, in order to send the dropped chopsticks to a compartment of the container 255, the chopsticks are placed in the chopstick storage portion 254, and then are dropped into the compartment of the container 255 while the chopstick storage portion 254 is rotated.

The chopsticks dropped into the compartment of the container 255 wait in front of the container belt automatic opening and closing portion 256 as the container 255 moves.

Here, the second hydraulic automatic opening and closing portion 251, the chopstick rotation portion 252, the chopstick automatic opening and closing portion 253, and the chopstick storage portion 254 are controlled by a second control portion 260.

In the rotary type inspection portion 300, when the chopsticks waiting at the container belt automatic opening and closing portion 256 are placed in the rotary type inspection portion 300, the chopsticks are fixed by a fixation hydraulic portion 310 so that the chopsticks do not slip out. At this time, after whether the chopsticks are positioned forward or reversed is determined by the image analysis portion 400, a rotation portion 320 is rotated to align the chopsticks so that the chopsticks can be moved in a correct direction (the same direction), then the chopsticks are sent to the packaging portion 500 when the chopsticks are in a normal state, and the chopsticks are sent to a defective product collection box 510 when the chopsticks are in an abnormal state.

Meanwhile, the spoon waiting at the spoon entrance 154 is fixed by the fixation hydraulic portion 310 when the spoon is placed on the rotary type inspection portion 300. At this time, a sanitary condition of the spoon is checked through the image analysis portion 400, then the spoon is sent to the packaging portion 500 when the sanitary condition is good, and the spoon is sent to the defective product collection box 510 when the sanitary condition is poor.

Here, the image analysis portion 400 may photograph foreign substances on the spoon by a camera, and may determine the good or poor conditions by using artificial intelligence and may separate and send the spoon.

In the packaging bag storage portion 600, a packaging bag 10 that can contain a spoon and chopsticks (hereinafter, referred to as the spoon and the chopsticks) is stored.

The packaging bag 10 stored in the packaging bag storage portion 600 is sent down and is stacked on a cover portion 610, and when one sheet of the packaging bag 10 is sent down through a packaging bag opening and closing portion 620, the packaging bag 10 is caught on a packaging bag fixing portion 630. At this time, when air is blown by an air compressor 640 and an opening of the packaging bag 10 is opened and then a set of the spoon and the chopsticks is dropped into the packaging bag 10 and packaged, the set of the spoon and the chopsticks is contained in a spoon and chopstick set storage container 520.

The operation portion 700 is configured such that a spoon and chopstick packaging quantity, a reservation of spoon and chopstick packaging quantity, a UV LED operation time, a hot air blower operation time, and so on are capable of being selected on a touch display, and a packaging bag quantity is capable of being displayed on the display, but is not limited thereto.

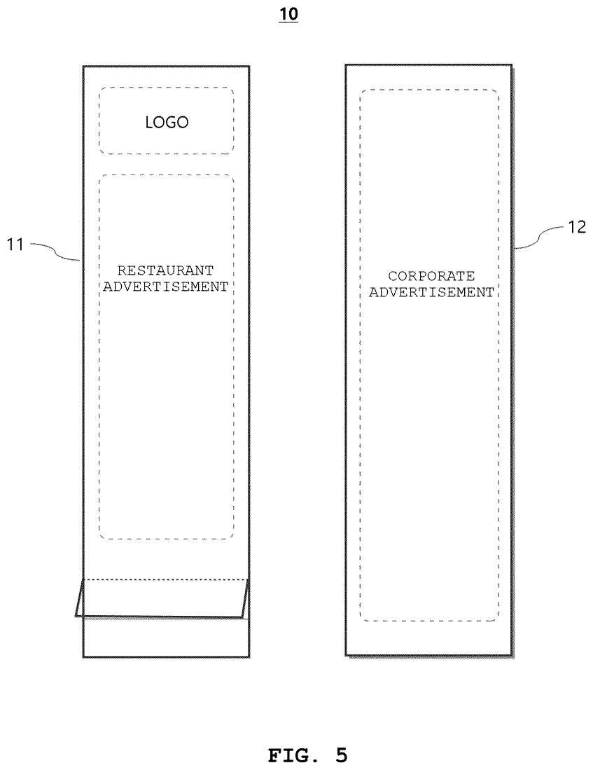

FIG. 5 is a view illustrating a front surface and a rear surface of a packaging bag that includes advertisements according to the present disclosure.

Referring to FIG. 5, a logo, a restaurant advertisement, and a corporate advertisement are printed on a front surface 11 and a rear surface 12 of the packaging bag 10, but is not limited thereto.

Although the embodiments of the present disclosure have been described above in detail, the scope of right of the present disclosure is not limited thereto, and various changes and modifications by those skilled in the art using the basic concept of the present disclosure defined in the following claims are also included in the scope of right of the present disclosure.

What is claimed is:

1. A spoon and chopstick automatic packaging apparatus, the apparatus comprising:
a spoon tray supply portion configured to supply a spoon tray in which a spoon is mounted in a groove formed in a spoon shape;
a chopstick tray supply portion configured to supply a chopstick tray in which chopsticks are aligned and contained;
a spoon moving portion configured to send down the spoon of the spoon tray so that the spoon waits at a spoon entrance;
a chopstick moving portion configured to send down the chopsticks of the chopstick tray so that the chopsticks wait at a container belt automatic opening and closing portion;
a rotary type inspection portion in which the spoon and the chopsticks are placed through the spoon moving portion and the chopstick moving portion;
an image analysis portion configured to check a sanitary condition of the spoon placed in the rotary type inspection portion and to check a direction of the chopsticks placed in the rotary type inspection portion;
a packaging portion configured to package the spoon and the chopsticks in a separate packaging bag; and
a packaging bag storage portion in which the packaging bag capable of containing the spoon and the chopsticks is stored.

2. The apparatus of claim 1, further comprising first and second hot air blowers, the first and second air blowers being respectively provided on opposite side surfaces of the spoon tray supply portion and the chopstick tray supply portion and being respectively configured to dry the spoon and the chopsticks contained in the spoon tray and the chopstick tray.

3. The apparatus of claim 1, further comprising first and second ultrasonic vibrators, the first and second ultrasonic vibrators being respectively provided on lower surfaces of the spoon tray supply portion and the chopstick tray supply portion and being respectively configured to spray sterilizing solutions of first and second sterilizing solution storage containers in a mist type to the spoon and the chopsticks in the spoon tray and the chopstick tray.

4. The apparatus of claim 1, wherein the spoon moving portion comprises:
a first hydraulic automatic opening and closing portion configured to send down the spoon of the spoon tray to a rotation frame portion;
the rotation frame portion configured to send down the spoon one by one to a guide portion by a first control portion so as to prevent the spoon from being overturned or reversed; and
a spoon entrance which is provided at a lower end of the guide portion and at which the spoon waits.

5. The apparatus of claim 4, wherein a first UV LED is mounted on a passage of the guide portion, and the spoon is sterilized by the first UV LED.

6. The apparatus of claim 1, further comprising a second UV LED, the second UV LED being mounted on an inner side surface of the chopstick tray supply portion storage portion and being configured to sterilize the chopsticks contained in the chopstick tray.

7. The apparatus of claim 1, wherein the chopstick moving portion comprises:
a second hydraulic automatic opening and closing portion configured to send down the chopsticks sterilized of the chopstick tray to a chopstick rotation portion;
the chopstick rotation portion configured to align the chopsticks passing through the second hydraulic automatic opening and closing portion and to send down the chopsticks in a line while rotating the chopsticks to prevent mixing of the chopsticks;
a chopstick automatic opening and closing portion configured to be closed immediately after dropping one pair of chopsticks;
a chopstick storage portion configured to send the dropped chopsticks in line with a compartment of a container rotated; and
the container belt automatic opening and closing portion at which the chopsticks moved through the container wait.

8. The apparatus of claim 1, wherein when the spoon and the chopsticks that are respectively waiting at the spoon entrance and the container belt automatic opening and closing portion are placed in the rotary type inspection portion, the spoon and the chopsticks are fixed by a fixation hydraulic portion.

9. The apparatus of claim 1, wherein, after whether the chopsticks placed in the rotary type inspection portion are positioned forward or reversed is determined, a rotation portion is rotated to align the chopsticks so that the chopsticks are capable of being moved in a correct direction, then the chopsticks are sent to the packaging portion when the image analysis portion determines that the chopsticks are in a normal state, and the chopsticks are sent to a defective product collection box when the image analysis portion determines that the chopsticks are in an abnormal state.

10. The apparatus of claim 1, wherein a sanitary condition of the spoon is checked through the image analysis portion, then the spoon is sent to the packaging portion when the sanitary condition is good, and the spoon is sent to a defective product collection box when the sanitary condition is poor.

11. The apparatus of claim 10, wherein the image analysis portion is configured to photograph foreign substances on the spoon by a camera, and is configured to determine the good and poor conditions by using artificial intelligence.

12. The apparatus of claim 1, wherein at least one of a logo, a restaurant advertisement, and a corporate advertisement is printed on a front surface and a rear surface of the packaging bag.

13. The apparatus of claim 1, wherein an individual packaging performed in the packaging portion is performed such that the packaging bag stored in the packaging bag storage portion is sent down and is stacked on a cover portion, and when one sheet of the packaging bag is sent down through a packaging bag opening and closing portion, the packaging bag is caught on a packaging bag fixing portion, and when air is blown by an air compressor and an opening of the packaging bag is opened and then a set of the spoon and the chopsticks is dropped into the packaging bag and packaged, the set of the spoon and the chopsticks is contained in a spoon and chopstick set storage container.

14. The apparatus of claim 1, further comprising an operation portion configured such that a spoon and chopstick packaging quantity, a reservation of spoon and chopstick packaging quantity, a UV LED operation time, and a hot air blower operation time are selected on a touch display and a packaging bag quantity is displayed on the touch display.
